(19) United States
(12) Patent Application Publication
(10) Pub. No.: US 2018/0362921 A1
(43) Pub. Date: Dec. 20, 2018

(54) EXTRACTION AND PROCESS FOR ACTIVE THYLAKOID MEMBRANES

(71) Applicant: GROUPE SANTÉ DEVONIAN INC., Montmagny (CA)

(72) Inventors: André P. BOULET, Blainville (CA); Nathalie BOUCHER, Trois-Rivières (CA)

(21) Appl. No.: 16/062,059

(22) PCT Filed: Dec. 13, 2016
(86) PCT No.: PCT/CA2016/051467

§ 371 Date: Jun. 13, 2018

Related U.S. Application Data
(60) Provisional application No. 62/266,770, filed on Dec. 14, 2015.

(51) Int. Cl. C12N 5/04; C07B 63/00

(52) U.S. Cl. CPC C12N 5/04; A61P 39/06; C07B 63/00

(57) ABSTRACT

There is provided an improved extraction process for obtaining integral & active thylakoid membranes that have longer shelf-life at room temperature. Particularly, the extract is lyophilized in a solution of PVP and remains stable with at least 80% of its original activity for at least 7 days at room temperature, respectively.

### FIELD OF THE INVENTION

This invention relates to the isolation and recovery of thylakoids, which are present substantially in their integral and natural state, at least a portion of which is functional or activatable. This invention further relates to the extraction of functional thylakoid membranes that are highly stable and remain active, particularly at room temperature, longer than membranes preserved by prior art methods.

### BACKGROUND

Antioxidants have become increasingly popular, namely in the biomedical field, because of their capacity to prevent the formation and the noxious activity of reactive oxygen species (ROS). Plants and other photosynthetic organisms are particularly well adapted to resist the effect of ROS, because of their efficient electron transfer mechanism through photosynthetic organelle membranes called thylakoids.

Chlorophyll is used by all photosynthetic organisms as the link between excitation energy transfer and electron transfer. All the chlorophyll in oxygenic organisms is located in thylakoids and is associated with PS II, PS I, or with antenna proteins feeding energy into these photosystems. PS II is the complex where water splitting and oxygen evolution occurs. Electron transfer, through PS II and PS I, results in water oxidation (producing oxygen) and NADP reduction, where the energy for this process provided by light. The initial electron transfer (charge separation) reaction in the photosynthetic reaction center sets into motion a long series of redox (reduction-oxidation) reactions, passing the electron along a chain of cofactors and filling up the “electron hole” on the chlorophyll, much like in a bucket brigade.

In eukaryotes (plants and algae), these thylakoids are located in chloroplasts and often are found in membrane stacks (grana and lamellae). Thylakoid organization is very sophisticated to extract the energy from light, and to transfer this energy to a proper location, and/or dissipate the same. The transfer is rendered possible and efficient by separating electrical charges and a high capacity to regenerate a neutral electrical state, ready for undertaking again a change in charges.

Chlorophylls are the main active pigments in thylakoids. The carotenoids have more than one role, depending on their location. A first role is as light collectors, which results in energy transfer from carotenoids to chlorophylls. A second role is as photoprotector, this time the energy transfer occurring in an opposite direction between chlorophylls and carotenoids. The transfer of energy is efficient only in conditions in which the pigments are very close to each other and in a specific organisation. The pigments have a specific organisation which should be preserved upon isolation and purification of thylakoids if the maintenance of the function of the latter is sought. It is therefore very important not to disturb the natural organisation of the pigments, keeping the membranes in an integral state, if one wants to purify active or fully activatable thylakoids.

One advantage of recovering intact thylakoids is found in their capacity to handle ROS. Such ROS are intended to cover free radicals (including super oxides), as well as nonradical oxidants such as singlet oxygen (1O₂) and peroxides. To obtain an extract that is optimally active, it is preferable to take every possible measure to maintain both pigments (chlorophyll and carotenoid) in their fundamental state. Isolated carotenoids, e.g. carotenoids not organized in thylakoid structures, would not be capable of an efficient quenching of triplet chlorophyll molecules. The advantage of having organized pigments is that the extract will retain the dynamism of natural thylakoid membranes, which has the capacity to capture ROS, to transfer the energy and to return to a state capable of undertaking new activation cycles again. This dynamism and capacity to regenerate is unique to organized pigments.

WO 2001/049305 discloses a method for their extraction. However, there is still a need to develop new processes for the extraction of native, organized, active thylakoid membranes that possess long-term stability.

### SUMMARY OF THE INVENTION

The present invention aims at providing a simple process for obtaining an extract having functional thylakoids. The present invention also provides an extract comprising isolated active thylakoids with long term stability. The stabilized extract is essentially free of any electron donor which would activate the thylakoids. This extract remains substantially active at room temperature for at least 5 days.

Since the most abundant electron donor is water, the stabilized extract is therefore preferably water-free. Water can be chased by a solvent or by drying (such as lyophilization), for example an amphoteric solvent. This type of solvent does not dissolve or disintegrate the membrane structural components, and has the advantage of replacing water molecules, therefore preventing the formation of aggregates upon dissolution in an aqueous solution.

The stabilized extract has a longer shelf life with no substantial loss of activity, as long as no electron donor such as water is added thereto. The stabilized extract is rehydrated extemporaneously before use to start the activation. The activity of the thylakoids once activated, lasts much longer than any other known antioxidant, which indicates a certain level of regeneration of activity rather than immediate and complete exhaustion.

In accordance with a first aspect, there is provided a method of extracting thylakoid membranes from a plant, the method comprising the steps of: obtaining a plant tissue having a specific Fv/Fm ratio; disrupting the tissue in a medium having specific viscosity and pH to obtain a mixture of cell debris and thylakoids in a liquid phase; separating said debris from thylakoids; suspending and filtering the thylakoids recovering and pooling fractions with specific Fv/Fm; and removing water from said pooled fractions by carrying out lyophilization in a solution comprising polyvinylpyrrolidone (PVP).

In accordance with a further aspect, there is provided a method of extracting thylakoid membranes from a plant, the method comprising the steps of: obtaining a plant tissue having Fv/Fm ratio of at least about 0.7; disrupting the tissue in a medium having a viscosity between 1 and 1.3 and a pH above 5 and below 8 to obtain a mixture of cell debris and thylakoids in a liquid phase; separating said debris from thylakoids; suspending and filtering the thylakoids recovering and pooling fractions with Fv/Fm greater than about 0.7; and removing water from said pooled fractions by carrying out lyophilisation in a solution comprising polyvinylpyrrolidone (PVP).

In accordance with a further aspect of the invention, there is provided a composition comprising an active thylakoid extract, in admixture with PVP.

According to a further aspect of the invention, there is provided a composition comprising a substantially pure thylakoid extract in admixture with PVP, the extract comprising organized photosynthetic pigments selected from: chlorophyll A, chlorophyll B, lutein and carotene; wherein the chlorophyll A is at a ratio of at least 0.6 of total pigment content; whereby the substantially pure thylakoid extract is substantially stable at room temperature for at least about 5 days.

Other aspects and features of the present invention will become more apparent upon reading of the following non-restrictive description of preferred embodiments thereof, given by way of example only with reference to the accompanying drawings.

The contents of the documents cited in the present disclosure are incorporated by reference thereto.

### DETAILED DESCRIPTION

This invention will be described hereinbelow, referring to specific embodiments and the appended figures, the purpose thereof being to illustrate this invention rather than to limit its scope.

### DESCRIPTION OF THE FIGURES

FIG. 1. Flow diagram of Compound A substance manufacturing process.

FIG. 2. HPLC chromatogram showing pigment profile of the thylakoid extract of the invention.

FIG. 3 shows a standard inhibition curve for atrazine.

FIG. 4 shows the quality control chart for the required inhibition by atrazine on several distinct batches of thylakoid extract.

FIG. 5 shows the relative activity of the extract of the present invention as a function of time when lyophilized in different concentrations of PVP.

FIG. 6 shows the relative activity of a thylakoid extract at room temperature as a function of time when lyophilized in presence of 2% PVP (polyvinylpyrrolidone) compared to 1 or 2% PEG (polyethylene glycol).

FIG. 7 shows the total antioxydant capacity of different stabilized thylakoid extracts of the invention, when fresh (age 0) and aged 1 or 6 years old.

FIG. 8 shows the nitric oxide (NO) inhibition in RAW 264,7 cells, of different stabilized thylakoid extracts of the invention, aged 1 and 6 years old.

FIG. 9 shows the superoxide dismutase activity of different stabilized thylakoid extracts of the invention, aged 1 and 6 years old.

### ABBREVIATIONS AND DEFINITIONS

### Definitions

The term “about” as used herein refers to a margin of + or −10% of the number indicated. For the sake of precision, the term about when used in conjunction with, for example: 90% means 90%+1-9% i.e. from 81% to 99%. More precisely, the term about refer to + or −5% of the number indicated, where for example: 90% means 90%+1-4.5% i.e. from 86.5% to 94.5%. In a different context, the term “substantially” may mean the same thing as “about”.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps.

The terms “functional thylakoid extract” or “functional thylakoid” as used herein, means purified functional photosynthetic pigments in their thylakoid membrane environment (i.e. in their integral native state such that they can still be activated).

As used herein, an “antioxidant” is a substance that, when present in a mixture or structure containing an oxidizable biological substrate, significantly delays or prevents oxidation of the biological substrate. Antioxidants can act by scavenging biologically important reactive free radicals or other ROS (singlet oxygen, >>O2-, H₂O₂, *OH, HOCl ferryl, peroxyl, peroxynitrite, alkoxyl . . . ), or by preventing their formation, or by catalytically converting the free radical or other ROS to a less reactive species. The antioxidant of the present invention is considered as such if, when added to a cell culture or assay reaction, it produces a detectable decrease in the amount of a free radical, such as superoxide, or a nonradical ROS, such as hydrogen peroxide or singlet oxygen, as compared to a parallel cell culture or assay reaction that is not treated with the antioxidant. Suitable concentrations (i.e., efficacious doses) can be determined by various methods, including generating an empirical dose-response curve, predicting potency and efficacy of a congener by using QSAR methods or molecular modeling, and other methods used in the pharmaceutical sciences.

The term “thylakoid” is used herein and means to cover organized photosynthetic membrane components obtained from photosynthetic organisms, eukaryotic and prokaryotic. When the organism has chloroplasts, the thylakoids comprise the following membrane constituents: PSI I, cytochromes b₆ and f, PSI and the coupling factor. Where thylakoids integrity and functionality has been tested from plant material, it has been measured between two reference points: proximal to PSII and distal to the coupling factor. For certain applications, thylakoids do not need to be active although they are apparently integral. Such thylakoids are performing and at least as stable as any other antioxidant. Therefore, “active thylakoids” means thylakoids having the capacity to be activated upon hydration, as opposed to inactive thylakoids which are integral but which have been actively or passively inactivated. In this case, the reaction center is inactive although thylakoids structure is substantially preserved.

### Detailed Description of Particular Embodiments

The present invention relates to isolated thylakoids and a method for isolation of thylakoids, which constitute powerful antioxidant molecules having a scavenger activity towards ROS. This antioxidant composition is of natural origin. It has no known toxicity, nor adverse effect.

This antioxidant composition must be properly formulated to be achieve stability of the antioxidant activity over time, thus ensuring a reasonable shelf-life. Stabilization is performed by withdrawing electron donors (namely water molecules), which renders thylakoids quiescent. Thylakoids are activated by adding an electron donor (particularly through hydration). Water can be chased by a solvent for example an amphoteric solvent, or a surfactant and/or by drying. A surfactant such as propylene glycol has been previously tried, with mitigated success.

Therefore, the present invention also provides an extract comprising isolated thylakoids with improved stability. The stabilized extract is therefore preferably water-free. The stabilized extract is essentially free of any electron donor which would activate the thylakoids and remains active at room temperature for at least 5 days, or even for up to about 7 days.

Particularly, the stabilized extract remains active at 4° C. for long periods of time such as, for example, 2 months or more, such as 1 year, or even up to 6 years.

### Conditioning

A first step undertaken, before going through the steps for recovering the thylakoids in a crude suspension, may be a conditioning step. This conditioning is optional and permits to vary the compositions of the extracts. To optimize the levels of pigments in their non-activated state (namely chlorophyll and carotenoids), a conditioning step may be performed in the same conditions as the working conditions, e.g. under green light or in the dark. Under such circumstances, the chlorophylls are preferably in a singlet state while the carotenoids are preferably in a fundamental state. This way, when ready to use, the carotenoids will be activated and ready to take the energy coming from a triplet chlorophyll.

### Method of Extraction

In accordance with a particular embodiment of the method of the invention, there is provided a method of extracting thylakoid membranes from a plant, the method comprising the steps of: a) obtaining a plant tissue having Fv/Fm ratio of at least about 0.7; b) washing said tissue with sodium hypochlorite at about neutral pH; c) conditioning said washed tissue under light conditions between 565 and 575 nm or under dark conditions, and at a temperature under about 10° C.; d) disrupting said tissue in a medium having a viscosity between 1 and 1.3 and a pH above 5 and below 8 to obtain a mixture of cell debris and thylakoids in a liquid phase; d) separating said debris from thylakoids under centrifugation with an upper filter and recovering an upper filter pellet essentially consisting of thylakoids; e) suspending said pellet and filtering under Sephadex-G100, recovering and pooling fractions with Fv/Fm greater than about 0.7; and f) removing water from said pooled fractions by carrying out lyophilisation in a solution comprising polyvinylpyrrolidone (PVP) to recover said extract substantially free of electron donor; wherein said substantially pure thylakoid extract comprises organized photosynthetic pigments selected from: chlorophyll A, chlorophyll B, lutein and carotene; wherein chlorophyll A is at a ratio of at least 0.6 of total pigment content; whereby said substantially pure thylakoid extract is stable at room temperature for at least about 5 days.

When one starts with whole plant or plant tissues thereof, the first step of the extraction is a dispersing step such as a homogenization step. The plant tissues are, for example, pulverized mechanically. The mesophylium tissues (leaves or needles) may be cut into small pieces with the aid of a rotative knife such as that retrieved in a homogenizer or a commercial rotative cutter. Any means leading to the dissociation of the cellulosic material to uncover the thylakoids would be suitable.

Besides working under a light source which optimally minimize the light flux (green light, λ=500-600 nm), the working conditions would ideally comprise a working temperature of about 2 to 20° C., preferably less than 4° C., for the purpose of increasing the cell density and of preventing any degradation by enzymes. The working conditions also include hypertonic conditions using hypertonic agents such as sugars. These conditions achieve optimal viscosity and fluidity. A specific example of a homogenization buffer is as follows:

TABLE 1
Homogenization medium

| Volume, | | | Final |
|---|---|---|---|
| weight | Product | pH | Molarity |
| 6 ml | Tris Buffer (1M) | 7.0 | 20 mM |
| 50 ml | Sorbitol (2M) | | 330 mM |
| 1.5 ml | MgCl₂ (1M) | | 5 mM |
| 243.5 ml | H₂O | | |
| 300 ml | Total | | |

The pH of the solution can vary from above 5 to below 8, more preferably maintained at a near neutral value of 7-7.5.

Taking spinach as a reference plant, the ratio wet weight of plant leaf tissues (g):volume of buffer (ml) is of about 1:3. Thus, the above recipe is suitable for extracting thylakoids from 100 g of spinach. The plant is mixed with the buffer and homogenized for example, in a domestic blender for about 30 seconds. The plant source may vary, so does the medium volume. The buffer itself may be any one suitable for maintaining a near neutral pH. For example, the above Tris buffer may be replaced with an acetate or ascorbate buffer. Sorbitol may be added to preserve the integrity of the membrane and to insure a viscosity varying from about 1 to 1.3 and may be replaced by any other suitable sugar such as commercial saccharose, fructose or turbinado in a concentration achieving the same effect as 0.2 to 1.5 M (preferably 0.2-0.4 M) sorbitol. Sucrose 0.2-0.4 M would be an acceptable less expensive component. Buffer components such as MgCl₂, NaCl, ascorbic salt/acid are not believed to be necessary to the present process, but they may help recovery more activity or preserving the activity for a long period.

A near neutral pH was preferably selected for maintaining an optimal concentration of H⁺ ions. Sugars and pH are important parameters for preventing the dissociation of photosynthetic pigments. The density of cell fluids is maximized when working in a cool or cold environment, namely below 4° C. Low temperatures also may protect components from enzymatic degradation. All these homogenization conditions release the membrane structure from its organization in chloroplasts without substantially affecting the molecular structural organization of thylakoids. The chloroplasts are therefore disorganized without destroying or disintegrating the thylakoids. The surface of cell components without any cellulosic protection is thus increased.

It was convenient in the present process to use plant tissues directly in an extraction medium. However, if it becomes advantageous to use pure chloroplasts or a preparation enriched in chloroplasts or even preparation of other photosynthetic organisms having or not chloroplasts, it is feasible to do so. Cultured cells or tissues can also obviously replace whole plants.

It is worthwhile noting that the yield may vary depending on the volume of buffer that was selected and on the water content of the selected plant. For example, pine needles have an endogenous water content that is much less important than in the case of spinach leaves. For an equal wet weight of plant material, the volume of buffer should be increased for isolating thylakoids from pine needles, when compared to the spinach leaves, taking into account all the parameters of the above equation.

The crude extract thus obtained is then separated/fractionated as follows.

### Separation of Plant Debris

The homogenization step is followed by a separation step. Thylakoids are separated from cell debris and from soluble components, based on their different sedimentation coefficients. The sedimentation coefficient of thylakoids is superior to that of cell organelles. The thylakoids are centrifuged for 10 minutes at 10,000×g in mobile buckets. A centrifuge force of less than 10,000×g but superior to 3,000 g may be used, adjusting the centrifugation time accordingly. The optimal handiness for the thylakoid pellet is obtained at 10000-12000×g for 10 minutes. Any other speed and time achieving equivalent results may be adopted. Different speed and time are contemplated in a scaling up process.

During sedimentation, the thylakoids pass through a filter corresponding to; 0.002≤X≤0.2 wherein X is calculated by multiplying the opening per the wire diameter (all in millimeters). The cell debris and membranes are stopped by this filter in a superior portion of a centrifugation tube. Thus, the bottom pellet comprising the thylakoids is easily recovered and the pellet may be used immediately or may be further fractionated or stabilized for any future use. Of course, any other method of separation achieving the same result of isolating thylakoids could be used. For example, a density gradient like a sucrose gradient could be used.

### Separation of Thylakoid-Enriched Fractions

A chromatographic or affinity medium and method could be also used. Referring to the above specific method, it is conceivable that the gross and fine separation would not be achieved in one step in a large-scale process. Therefore, a gross purification could be made first on a press or a filter and fine separation of thylakoids and the liquid phase may be achieved in a later step, such as:

### Membrane Filtration

Size exclusion chromatography may be carried out to separate the most active fractions by molecular weight. Particularly, the crude thylakoids may be further purified on Sephadex G-100 filtration and the fractions corresponding to a ratio of Fᵥ/Fₘ of at least 0.7 are recovered.

### Thylakoid Integrity and Activity

The extract comprises substantially pure thylakoids (>90%); they are photosynthetically activatable; stable; and the extract is controllable. The photosynthetic activity has been evaluated with different techniques: oxygen release (Schlodder et al. 1999), photoreduction of 2,6 dichlorophenol indophenol (DCPIP) (Behera et al. 1983) and fluorescence (Maxwell et al. 2000). Moreover, the integrity of the thylakoids has been evaluated with a technique which measures a continuous electric current: any disorganization should be detected by any variation in this electric current. The current is measured from PSII to the coupling factor, which indicates that the thylakoids contain the main subunits listed above and that they are functional.

When a green light is used in the working conditions, the pigments are stabilized in their fundamental state (F0), thus, permitting the optimization and synchronization of any desired effect. The stabilization is possible because of the withdrawal of the primary electron donor. The stability measured by the photosynthetic activity (absent during quiescent state and present upon activation with an electron donor) and the concentration in chlorophylls and carotenoids, persist for several months after extraction. The ratio chlorophylls/carotenoids is also important for the activity of the complex and to maximize the absorption and dissipation of energy.

The extracts are easily detectable because of their natural fluorescence. No toxic product, solvent, detergent or conservation agent has been added to the above thylakoids, preserving all its original nature. The extracts are edible. Even when propylene glycol is used to stabilize the thylakoids, this solvent is harmless because its oxidation yields pyruvic and acetic acids. PVP is a non-penetrating agent, acting to improve the osmotic imbalance occurring during freezing. This solvent is currently used as a food emulsifier, which means that it has surfactant properties (however, non-deleterious to the integrity of the thylakoids). It further has an inhibitory activity against fermentation and mold growth.

### Stabilization

The separation step is followed by a stabilization step. This step allows withdrawal of electron donors such as water molecules that are bound or non-bound to membranes, thus eliminating any activator of the PSII system. The fractions having Fᵥ/Fₘ of 0.7 are recovered, pooled and placed in clean vials. The vials are then submitted to a vacuum drying at low temperature (about −20 to −50° C.) for at least 4 hours. The extracts so lyophilized remain activatable, more particularly at 4° C., until water is added thereto.

### Long Term Preservation of Activity Before Resuspension in Aqueous Medium

Polyvinylpyrollidone (PVP) has been found to advantageously provide longer shelf-life of the active extract when used alone for lyophilization, or when used in combination with sucrose or sorbitol.

PVP is an amphiphilic water-soluble polymer used to stabilize synthetic vesicles (liposomes) or biological membranes (such as thylakoids) by steric protection and increasing the viscosity of the solution lowering the rate of growth of ice crystals. This surfactant is also non-toxic.

In accordance with a particular aspect of the invention, there is provided a thylakoid extract made by the method as defined herein. According to an alternative aspect, there is provided a composition comprising a thylakoid extract combined with PVP prior to lyophilization. More particularly, the PVP is at a concentration ranging from about 0.5% to about 5% of the solution prior to lyophilization, more particularly: about 0.5, 1%, 2% and 5%, most particularly: about 2%.

According to a particular embodiment, the method of the invention yields a substantially pure thylakoid extract that is stable at 4° C. for at least about 2 months, particularly about 1 year while maintaining at least about 70%, more particularly about 80%, most particularly about 90% of its original ORAC activity.

According to a further particular embodiment, the method of the invention yields a thylakoid extract having an original NO inhibition activity at day 0, whereby said substantially pure thylakoid extract is stable at 4° C. for at least about 2 months, particularly at least about 1 year while maintaining at least 50% of its original NO inhibition activity when thylakoids are assessed at 0.25 mg/Ml.

In accordance with a particular embodiment, the method of the invention provides a thylakoid extract having an original SOD activity at day 0, whereby said substantially pure thylakoid extract is stable at 4° C. for at least about 2 months, particularly at least about 1 year, while maintaining at least about 60% of its original SOD activity.

More particularly, the extract is stable for about one year; most particularly for about 6 years.

In accordance with a particular embodiment, the invention provides composition comprising an active thylakoid extract, in admixture with a stabilizing concentration of PVP.

Particularly, the terms “stable activity” or “stability” means that the activity of the extract is substantially unchanged after at least 5 days at RT, the activity being selected from the group consisting of: antioxidant activity as measured by at least one of: ORAC, Fv/Fm activity, inhibition of NO production and SOD activity.

More particularly, the terms “stable activity” or “stability” means that the activity of the extract is substantially unchanged after 2 months, or one year, or even up to 6 years at 4° C., the activity being selected from the group consisting of: antioxidant activity as measured by at least one of: ORAC, Fv/Fm activity, inhibition of NO production and SOD activity. More particularly, the activity is at least about 70% of its original activity, more particularly about 80%, most particularly about 90% of its original ORAC activity.

In accordance with a particular embodiment, the invention provides a composition comprising an active thylakoid extract, having from about 130 to about 195 μmol Trolox equivalent per g of thylakoid (μmol ET/g).

In accordance with a particular embodiment, the invention provides a composition comprising an active thylakoid extract, having at least about 33% NO inhibition at a concentration at 0.25 mg/mL of thylakoid.

In accordance with a particular embodiment, the invention provides a composition comprising an active thylakoid extract, having an SOD activity of at least about 0.38 mU/mg prot/min, more particularly at least about 0.40, most particular at least about 0.50 mU/mg prot/min.

### Formulation for Use

The extracts may be presented in a solid form, dry or humid, or in a liquid form. However, it is important to note that thylakoids are reactivated upon rehydration. Therefore, the extracts should be kept in dehydrated form, as long as possible, before use such that their activity remains maximal. The extract is therefore resuspended in aqueous medium immediately prior use. Therefore, it may be better to separate each dose in a distinct aliquot that is suspended immediately prior use. However, since it may prove difficult or expensive to provide the extract as distinct aliquots (single doses), it may prove useful or convenient to provide several dosages in a single vial. In that instance, the activity of the aqueous extract will last for at least 5 days at RT, or longer at 4° C., prior to administration.

It is therefore an aspect of the present invention to provide a formulation for long term preservation of the activity of the thylakoid extract once resuspended in aqueous medium to ensure longer shelf life.

The following examples are put forth to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### EXAMPLES

### Example 1—Preparation of Thylakoid Extract

Thylakoids originate from the mesophyll tissue of baby spinach (Spinacia oleracea L.) leaves, which is rich in chloroplasts. The inner membranes of the chloroplasts, organized in structures known as thylakoids, are extracted from baby spinach, concentrated and stabilized into a solid powder form. The major constituents of thylakoid membranes are pigments, proteins and lipids.

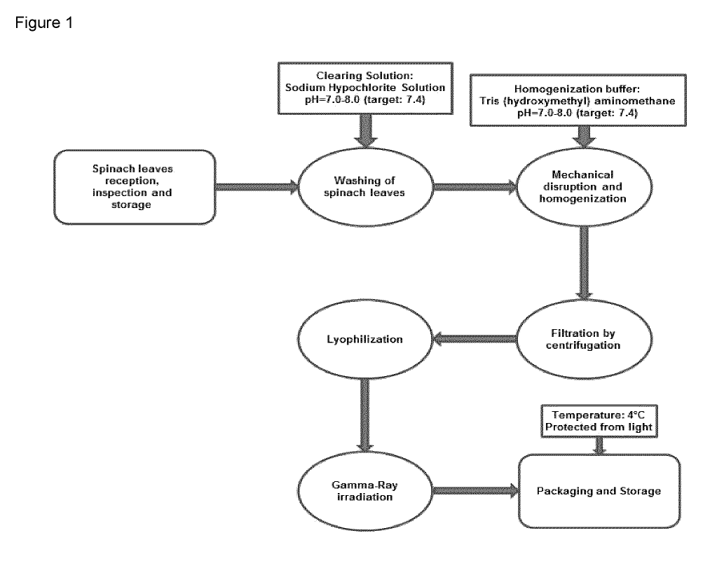

The extraction process for thylakoid extract is presented schematically in the flow diagram of FIG. 1. The processing steps were executed in the following order: inspection of spinach leaves and washing with a sodium hypochlorite solution; mechanical disruption and homogenization; filtration by centrifugation; filtration by Sephadex-100; lyophilisation; and gamma-ray irradiation.

Inspection of Spinach Leaves and Washing with a Sodium Hypochlorite Solution.

After visual inspection was performed to verify dimensional and identity attributes (e.g. green leaves without discoloured zones or yellowish pecks (chlorose)), spinach leaves were first washed at a fixed solution-to-leaves ratio (44 kg:5.4 kg) on a mass basis, with a sodium hypochlorite solution adjusted to a pH between 7.0 and 8.0 (target pH: 7.4) to reduce the microbial flora naturally found on the leaves of fresh produce.

### Mechanical Disruption and Homogenization.

After draining the excess sodium hypochlorite solution, leaves were transferred into a mechanical cutter/mixer along with a fixed volume of Tris (hydroxymethyl) aminomethane buffer solution at pH between 7.0 and 8.0 (target pH: 7.4) at a fixed solution-to-leaves ratio (5.4 kg:3.7 kg) on a mass basis. This stepwais used to cut and homogenize the leaves into a coarse suspension while freeing up fragments of the thylakoid membranes originating from chloroplasts.

### Centrifugation

The suspension was then filtered in a basket centrifuge. The centrifugation was performed at a target speed of 10000 rpm for about 10 minutes. This step allowed the removal of fibers, debris and coarse material which were retained on a screen, yielding a by-product cake that was discarded. The thylakoids were found in the centrifugate (pellet), were collected and kept at a temperature below 10° C. for further processing.

### Filtration

After centrifugation, the thylakoid extract was suspended in at least 3 times its volume of Tris-HCl 50 mM pH 7.5 and NaCl 50 mM and put on a Sephadex G-100 column equilibrated with the same buffer. The thylakoids were then eluted in the same buffer and each eluted fraction was tested for its Fᵥ/Fₘ ratio. Those representing an Fᵥ/Fₘ ratio higher than 0.7 were kept and pooled.

### Lyophilisation

### With Buffer Alone:

Thylakoids were frozen at −35° C. for 4 hrs and lyophilised at the same temperature for 72 hrs at a pressure of 100 m Torr. After lyophilisation, the thylakoids were grinded under vacuum and stored at different temperatures (RT, 4° C. or −20° C.) in accordance with the stability test (see Example 4).

### With Buffer and PVP:

PVP was added to the pooled thylakoid fractions. They were then frozen at −20° C. for 2 hrs and lyophilized under the following conditions: a) −20° C. for 48 hrs; b) −10° C. for 3.5 hrs; c) 0° C. for 1.5 hrs; and d)+20° C. for 18 hrs.

### Example 2—Pigment Composition and Other Characteristics

Spinach contains natural antioxidants (e.g. flavonoids) and photosynthetic pigments (chlorophyll and carotenoids). The inner membranes of the chloroplasts are organized in structures known as thylakoids. The major constituents of thylakoid membranes are pigments, proteins and lipids.

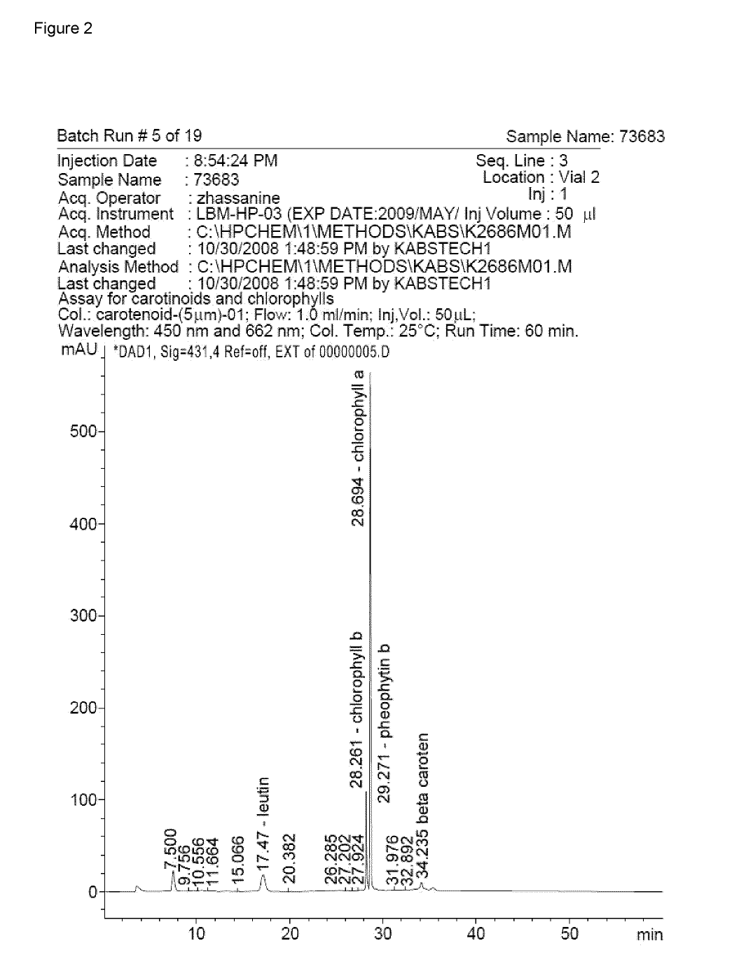

Thylakoids originate from the mesophyll tissue of spinach leaves which are rich in chloroplasts. To date, the following pigments have been identified in the thylakoid extract using HPLC analysis: lutein, chlorophyll b, chlorophyll a, pheophytin and β-carotene. A typical chromatogram showing the pigment profile of the thylakoid extract, in area %, is presented in FIG. 2. This analysis shows that the major constituent of the thylakoid extract is chlorophyll a (62.5%), followed by chlorophyll b (13.1%), lutein (9.4%), β-carotene (2.98%) and pheophytin (0.45%).

Preferably, raw baby spinach leaves were used from a grower certified as per the National Organic Standards of the United States Department of Agriculture (USDA) to minimize risks of presence of potential chemical residues from fertilizers or pesticides in the thylakoid extract.

### Justification for Specification

The thylakoid extract is characterized by its pigment content, which is expressed in milligram of pigment per gram of powdered extract. Based on process capabilities and allowing for seasonal variability in the herbal starting material, a specification of not less than 25 mg/g was set at release. Based on stability data gathered to date, a limit of 80% of the initial pigment content was set for shelf-life.

A pigment profile also allows identification of the various pigments present in the thylakoid extract and their ratios in area percent. Given the profile determined in batches manufactured to date, it has been established that chlorophyll a, chlorophyll b, lutein and β-carotene should be present and that the average ratio of chlorophyll a to total peak area response should not be less than 0.60 (FIG. 2).

Since water was used as extraction solvent in the manufacturing process, a test to determine water content in the thylakoid extract was included. A specification of not more than 10% w/w of water was set to control moisture.

TABLE 2
Thylakoid extract specifications

| | |
|---|---|
| Physical appearance: | Dark green powder |
| Solubility in water: | Insoluble |
| Solubility in alkaline medium (pH 10.6) | 0.5 mg/mL |
| Solubility in acidic medium (pH 1.0) | 0.3 mg/mL |

### Example 3—Stability Studies

Different batches of thylakoid extract were lyophilized in the same buffer but with different cryoprotectant, packaged in a jar with a tight screw cap, and placed on shelves, protected from light, at room temperature for at least 7 days. A first batch was lyophilized with buffer with PEG as previously disclosed (WO 01/049305). A second batch was lyophilized with PVP.

Thylakoid extracts were lyophilized in the following buffer: Tris-HCl 50 mM pH 7.5; Sorbitol 330 mM; MgCl₂ 2 mM; with added PEG at 1-2% or with PVP (0.5% to 5%) and then stored at −20° C. until further use.

To determine their stability at room temperature after rehydration, both lyophilized thylakoid extracts were resuspended in: Tris-HCl 50 mM pH 7.5; sorbitol 330 mM; MgCl₂ 2 mM at 5 μg/mL of chlorophyll as measured according to Porra et al., 1989. Each re-suspended extract was dark-incubated for 15 min with the appropriate atrazine concentration for 15 minutes.

### Inhibition of Fv/Fm

Dark-adapted conditions allow the inhibition of photochemistry and complete re-oxidation of PSI electron acceptors and opening of PSII reaction centers). When thylakoids are then re-exposed to light, the absorbed light can then be maximally used for photochemistry.

Absorbed light was measured with a FMS1 Hansatech Instrument (Schreiber et al., 1986; lab based modulated fluorescence); under excitation light beam at 470 nm. In dark adapted condition, the following fluorescence parameters can be obtained or calculated:
Fo: minimal fluorescence when PSII reaction centers are in the fully oxidized state. It is obtained at the beginning of fluorescence measurement when only the modulating beam is illuminated.
Fm: obtained with an intense saturating pulse (2 000 μmol photons m²/s) allowing maxi mum PSII fluorescence emission.
Fv: variable fluorescence is obtained by the difference between Fm and Fo and corresponds to maximum capacity for photochemical energy quenching.

Fv/Fm: this ratio is proportional to quantum efficiency of PSII reaction centers (Butker 1977, 1978) and represents a correlation between the chlorophyll fluorescence and the photochemical reactions (for instance oxygen evolution). It is widely used as a screening parameter for stress response (Björkman and Demmig 1987). Preferably, the photosynthetic efficiency of the thylakoids is stable over time at Fv/Fm of 0.7±0.1 (0% inhibition of atrazine).

Stability is referred to as the stability of a % inhibition obtained at a specific atrazine concentration or as the stability of the IC₅₀ (atrazine concentration inhibiting 50% of the efficiency parameter).

TABLE 3
Parameters for determining photosynthetic stability of thylakoids

| | |
|---|---|
| Control efficiency | efficiency = Fm − Fo/Fm(Fv/Fm) |
| (without inhibiting molecule) | |
| Sample efficiency | efficiency (sample) = Fm(sample) − |
| (with inhibiting molecule) | Fo(sample)/Fm(control) |
| Percentage efficiency | efficiency (%) = (efficiency(sample) × |
| | 100)/efficiency(control) |
| Percentage inhibition | Inhibition (%) = 100 − efficiency (%) |

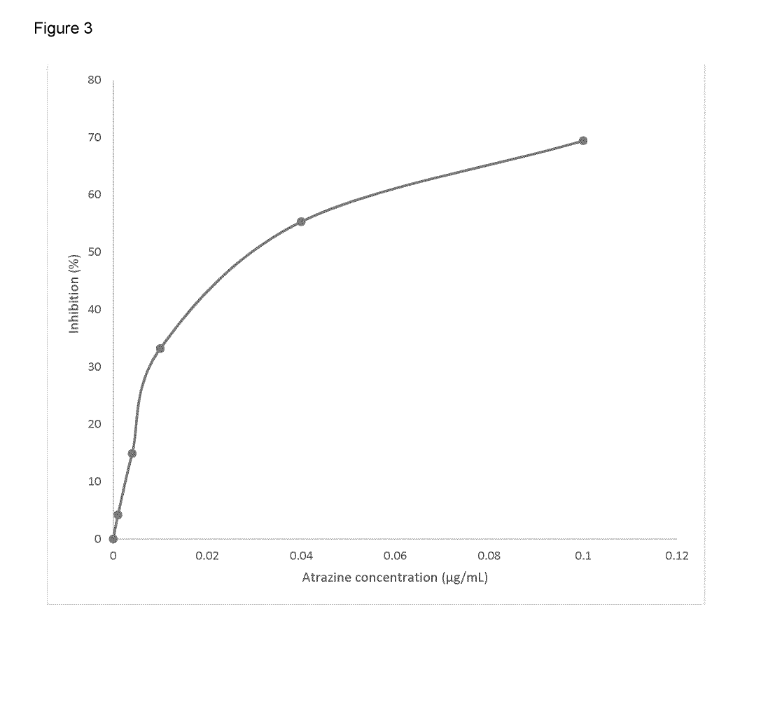

Any molecule affecting the photosystems will modify either Fo or Fm which modification necessarily disrupts (increases or decreases) the photosynthetic efficiency. The inhibition curve of a particular molecule was determined by calculating the efficiency ratio of the photosystems in the absence and in the presence of the molecule and comparing both efficiency as follows (described in Conrad 1993). The inhibition curve was measured with atrazine after the extract had spent 7 days at 20° C., and the IC₅₀ was determined as the concentration required to obtain 50% inhibition (FIG. 3).

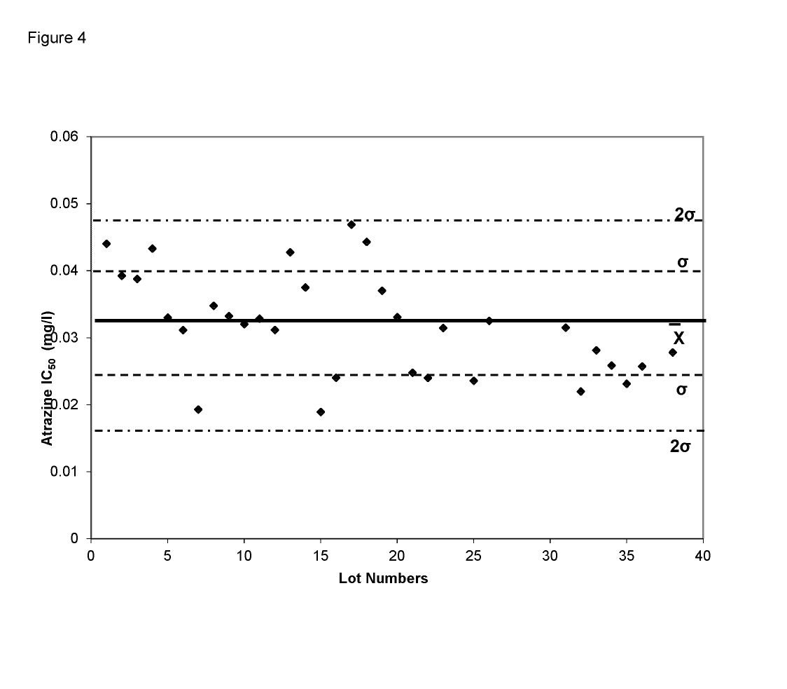

IC₅₀ had to be comprised between 2σ of the mean IC₅₀ (for 95% of confidence) for a lyophilization to be released. Therefore, the IC₅₀ of an accepted lot must be between 0.0165-0.0470 μg/mL (FIG. 4). In the present case, the atrazine concentration was determined to be 0.025 μg/ml.

### Stability Comparison

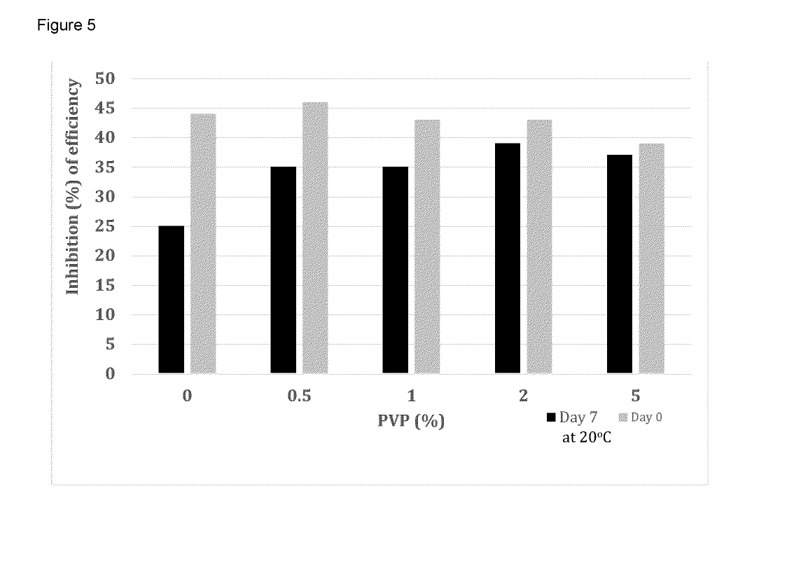

Stability study was carried out for batches lyophilized with added PEG or PVP. Thylakoids were stored at 20° C. under vacuum and dark after lyophilization. The inhibition (% of efficiency) was given by the % of inhibition obtained with 0.025 μg/mL of atrazine. FIG. 5 shows that increasing concentrations of PVP increase the stability of % Fv/Fm inhibition at Day 7. The stability was optimized with 2% PVP concentration in the lyophilization buffer yielding a retention of about 84% of its original activity after 7 days at room temperature.

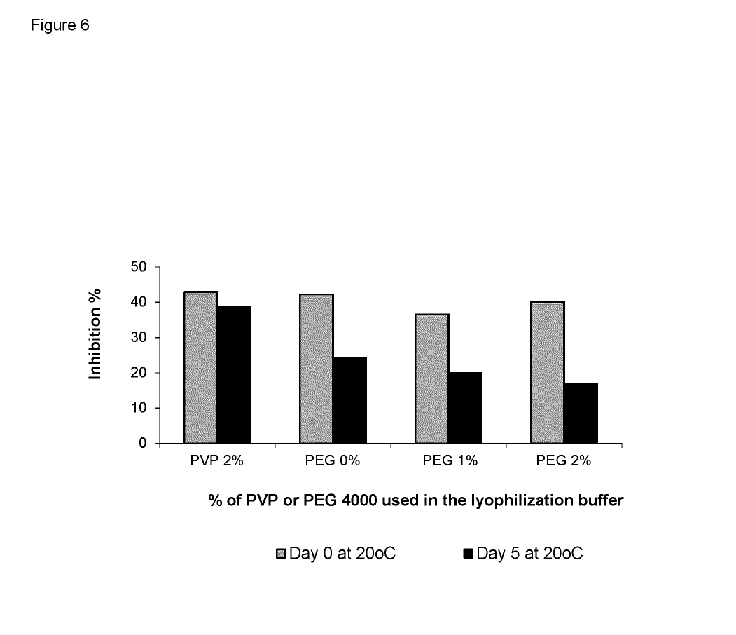

The % inhibition was also evaluated with 0.025 μg/mL atrazine when concentrations of 1% or 2% of PEG was added to the lyophilization buffer. After lyophilization, thylakoids were stored at 20° C. under dark and vacuum conditions. Fv/Fm inhibition was measured immediately after lyophilization (Day 0) and after 5 days at 20° C. (Day 5). Surprisingly, FIG. 6 shows that PEG does not provide the same retention of activity after 5 days at 20° C. than the one measured when 2% PVP is added to the lyophilization buffer. In fact, 2% PVP in the lyophilization allows the extract to retain about at least 90% of its original activity after 5 days at RT.

As shown in Table 4, when PVP is added to the lyophilization buffer, the stability of the hydrated solution was unexpectedly longer than many other stabilization methods previously disclosed:

TABLE 4
Comparison of thylakoid stability stabilized with different methods

| | Thylakoid Stability at |
|---|---|
| Stabilization method | 20-22° C. |
| Immobilisation in a Glutaraldehyde- | 2 days |
| Albumin Matrice¹³ | |
| Immobilization in a Poly(vinylalcohol) | 1 day |
| bearing styrylpyridinium groups¹⁴ | |
| Thylakoids coupled to a screen printed | 1 day |
| electrode¹⁵ | |
| 1% Polyethylene glycol (PEG) in | 48.7% activity |
| lyophilization buffer | remaining at 5 days |
| Polyvinyl pyrrolidone | Maintenance of at least |
| (PVP) in lyophilization | about 70% activity for |
| buffer (this invention) | 7 days at RT |
| | (>80% for 2% PVP) |

### Example 4—Stability Over Time of Antioxydant Capacity of Thylakoid Extract

Pigments found in thylakoid membranes provide a high level of antioxidant capacity/activity. This activity was tested on thylakoid extracts stabilized as described in the present invention. The oxygen radical absorbance capacity-fluorescence test (ORAC_{FL}) was assessed in fresh thylakoid extract and in samples aged 1 and 6 years old, to compare their respective stability of antioxidant capacity.

### ORAC_{FL} Test

Lipophilic and hydrophilic antioxidant capacities were determined on thylakoid extract powder using the ORAC_{FL} test (from fluorescein as fluorescent probe and 2,2′-azobis (2-amidinopropane) dihydrochloride (AAPH) as generator of peroxyl radicals. In presence of free radicals generated by AAPH, the hydrophilic and hydrophobic fractions of thylakoids protect the fluorescent probe gradually inhibited by the peroxyl radicals. The higher the antioxydant capacity of the extract, the more the fluorescein remains fluorescent. The results of antioxidant capacity are defined in relation to the antioxidant capacity of a reference molecule: Trolox.

Fluorescence was read with a FLUOstar Galaxy plate reader (BMG Lab Technologies, Durham, N.C.) equipped with a fluorescence filter providing excitation and emission wavelengths at 485 and 520 nm, respectively (Microplates 96-wells).

### Preparation of Samples and Hydrophilic and Hydrophobic Fractions

Thylakoid extracts were extracted manually: 1 gram of sample was placed in presence of hexane/dichloromethane (1:1 Hex/Dc), followed by acetone/water/acetic acid (70/29.5/0.5). The latter fraction represented the hydrophilic or aqueous fraction whereas the fraction resulting from Hex/Dc mixture constituted the hydrophobic or lipophilic moiety.

Fractions Hex/Dc were dried under nitrogen atmosphere in a water bath at 30° C., and the residue was reconstituted with 10 ml of acetone:water, containing B-cyclodextrin. After centrifugation, the supernatant was used to measure the lipophilic ORAC_{FL} following further dilution with assay buffer, if necessary. The hydrophilic fractions were transferred into a volumetric flask of 25 ml and diluted with 25 ml acetone/water/acetic acid (70/29.5/0.5) (total volume). This solution was used to measure the hydrophilic ORAC_{FL} fraction. Each sample was extracted and tested in duplicate.

Both hydrophilic and lipophilic ORAC assays were performed on a FLUOstar Galaxy plate reader. AAPH was used as peroxyl generator and Trolox as the reference. The final ORAC_{FL} values were calculated using a quadratic regression equation (y=ax²+bx+c) between Trolox or sample concentration and the net area under the fluorescein decay curve. Data were expressed in micromoles equivalent Trolox (ET) per gram of sample (μmol ET/g). Total antioxidant capacity (TAC) was calculated by adding the results of hydrophobic and hydrophilic ORAC.

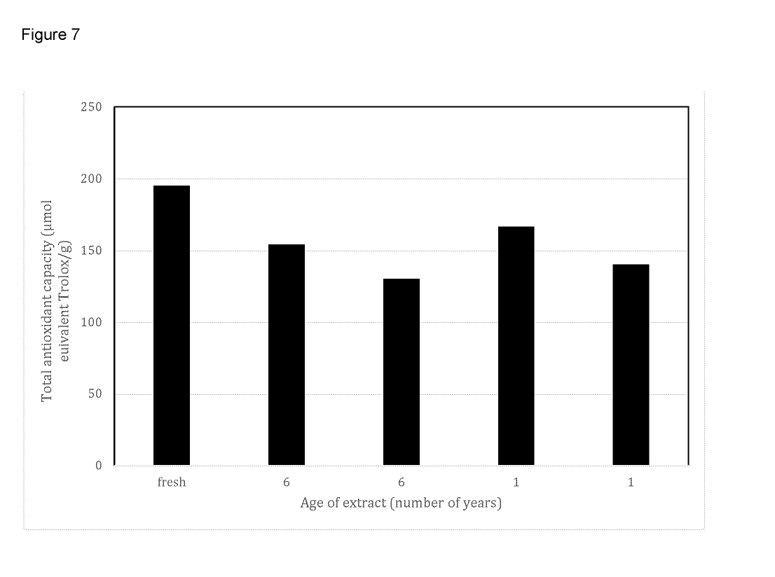

The results of FIG. 7 demonstrate that the antioxidant capacity remains at, or higher than, about 70% of its original activity for at least 6 years after extraction of the thylakoid membranes. In thylakoid extracts stabilized in PVP, the total antioxidant capacity varied from about 135 to 165 μmol of equivalent Trolox/g (μmol ET/g) of thylakoid extracts.

### Example 5—Stability Over Time of NO Production of Thylakoid Extracts

The same thylakoid extracts as those used in Example 4 were tested for their effect on NO production. The stabilized thylakoid powder extracts of the present invention were reconstituted at 5 mg/mL in Hank's buffer.

### Murine Cell Culture

Murine macrophage-like cells RAW 264,7 is one of the most widely used cell line to investigate the function and differentiation of monocytes and macrophages in response to various inflammatory mediators. RAW 264,7 is a macrophage-like cell model that produces large amounts of NO in response to INF-γ, TNF-α, bacterial infection or bacterial products, such as LPS. In this experiment, RAW 264,7 cells were maintained in RPMI-1640 medium supplemented with 10% heat-inactivated bovine serum containing 1 mM sodium pyruvate, 10 mM HEPES and 50 μg/mL gentamycin, at 37° C. in a moisture-saturated atmosphere containing 5% CO₂.

### Evaluation of NO Production by the Griess Reagent Method:

RAW 264,7 cells (25×10³ cells/well) were grown and pretreated with various reconstituted thylakoid extracts at concentrations of protein of 2.5 μg/mL and 1 mg/mL. After pretreatment, cells were washed twice with 10% FBS RPMI-1640 and then activated to produce NO for a period of 24 h. NO production was measured using the Griess reagent method involving the detection of nitrite ions (NO₂⁻) formed by the spontaneous oxidation of NO under physiological conditions. Equal volumes of sulfanilic acid and N-(1-naphthyl) ethylenediamine are mixed together to form the Griess reagent. In the presence of NO₂⁻, sulfanilic acid is converted to a diazonium salt, which in turn is coupled to N-(1-naphthyl) ethylenediamine to produce a pink coloration that is measured with a spectrometer at 548 nm. NO concentration is expressed in μM.

### Inhibition of Nitric Oxide (NO) Production

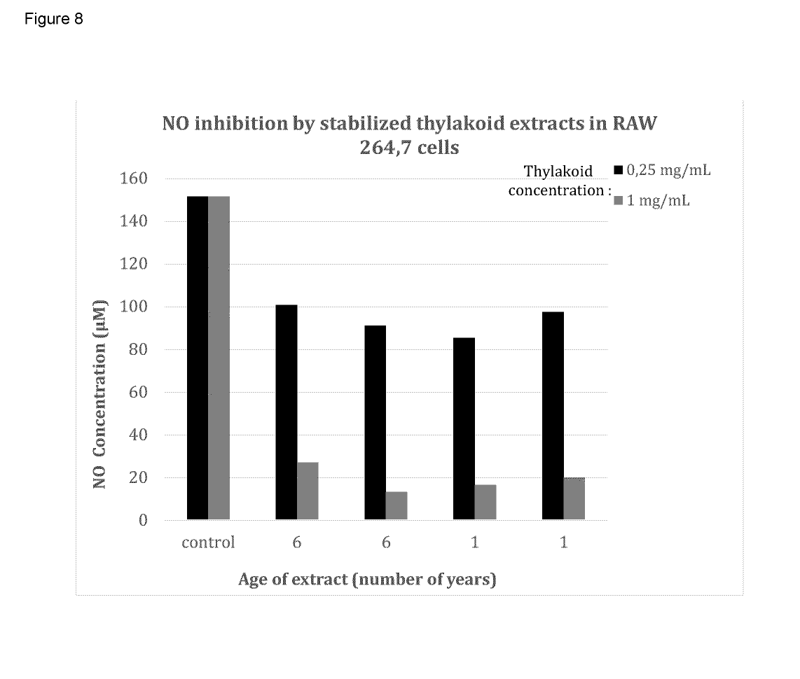

NO is a key mediator of immunity by regulating immune responses. In association with reactive oxygen species (ROS), it triggers the eradication of pathogens. NO and ROS can also modulate immunosuppression. The effect of stabilized thylakoids on NO production was tested on murine cells. The results are presented in FIG. 8. It can be observed that untreated murine cells (control) produce 150 μM of NO. This production is reduced to about 80-100 μM NO (from 33% up to almost 50% inhibition) when murine cells are treated with 0.25 mg/mL of thylakoid extracts and to about 15-28 μM NO when murine cells are treated with 1 mg/ml of thylakoids. The effect of thylakoids on NO production is stable over time, thylakoids extracts aged 1 and 6 years having substantially the same inhibitory activity on NO production.

### Example 6—Stability Over Time of SOD Activity of Thylakoid Extracts

Reactive oxygen species (ROS) are free radical derivatives of oxygen. The best-known ROS include superoxide anion (O₂), hydrogen peroxide (H₂O₂) and the hydroxyl radical (OH⁻). They are constantly produced in the body during various metabolic activities (cell respiration and photosynthesis and by various exogenous factors (sunlight, air pollution, UV light, ionizing radiation). Living organisms have developed an antioxidant system to countervail the activity of oxidation-reduction system, of which SOD is the master antioxidant enzyme since it scavenges superoxide (O₂⁻, the first ROS produced by the oxidation-reduction system of the cell) to produce H₂O₂. A cascade of reactions follows to neutralize H₂O₂ and other ROS.

### SOD Assay

SOD activity was assessed using photo-oxidation of riboflavin as a ROS-generating reagent. Riboflavin is a reliable substrate and is exploited in several studies to stimulate light-dependent superoxide production that is rapidly converted to H₂O₂ by SOD. The method using indirect assay comprises several reactions: the photochemically excited riboflavin is first reduced by methionine into a semiquinone, which donates an electron to oxygen to form the superoxide source. The superoxide converts Nitro Blue Tetrazolium (NBT) into a blue formazan product that is measured at A₅₆₀ₙₘ. In this way, the SOD activity is inversely related to the amount of formazan produced.

In the following protocol, superoxide dismutase (SOD) activity was assessed by its ability to inhibit photochemical reduction of NBT at 560 nm. Thylakoids (0.1 g) were re-suspended in 10 mL of extraction buffer (50 mM potassium phosphate buffer (pH 7.8), 1 mM EDTA and 2% (w/v) PVP). The suspension was centrifuged 30 min at 14 000 rpm and 4° C. and the supernatant was assessed for SOD activity.

The reaction mixture for the SOD assay contained: 20 μL of supernatant, 180 μL of extraction buffer, 1.3 mL of assay buffer (50 mM K—PO₄ buffer (pH 7.8), 1 mM NBT, 500 mM L-methionine, 10 mM EDTA and 2.5% (v/v) Triton,). This mixture was kept in the dark until the assay substrate, riboflavin (0.2 mM) was added. The reaction started by illuminating the reaction mixture containing riboflavin with a luminescent lamp 5 minutes at room temperature. The sample was then read at 560 nm. A standard curve with bovine SOD was carried out (SOD enzymatic units over % activity) and was used to determine the SOD activity of different thylakoid extracts. The results were expressed as mU of SOD/g of total proteins in thylakoid extract/minute. Protein content in the different thylakoid extracts was determined by the Bradford method.

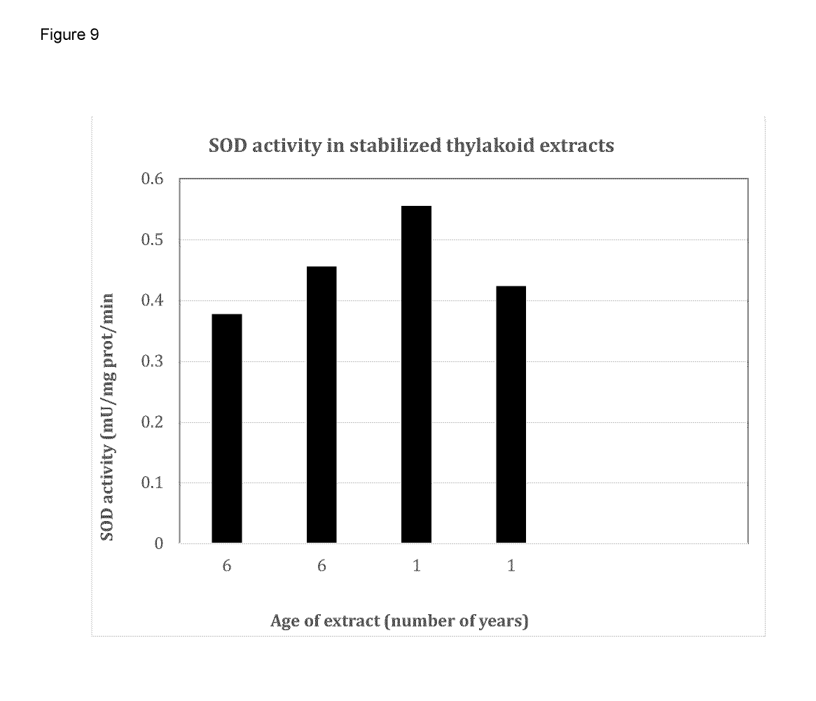

FIG. 9 shows that SOD activity found in thylakoid extract is about 0.4 to 0.5 mU/mg protein/minute. The most active extract reached about 0.56 mU/mg protein/minute and was obtained for a thylakoid extract aged one-year old. Even if the SOD activity ranged from 0.38 to 0.56 mU/mg protein/minute, the number of years of thylakoid extracts does not seem to significantly modify this activity since the two other extracts aged 1 and 6 years demonstrated about 0.38 and 0.45 mU/mg protein/minute, respectively.

These results obtained from different thylakoid extracts of varying ages show that the extraction and stabilization method of the present invention keeps thylakoid membranes in their structural integrity allowing them to be active, even after 6 years at 4° C.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features herein before set forth, and as follows in the scope of the appended claims.

All patents, patent applications and publications mentioned in this specification are herein incorporated by reference to the same extent as if each independent patent, patent application or publication was specifically and individually indicated to be incorporated by reference.

### REFERENCES

1—Schlodder E, Witt H T. (1999). Stoichiometry of proton release from the catalytic center in photosynthetic water oxidation. Reexamination by a glass electrode study at ph 5.5-7.2. J. Biol. 274 (43): 30387-30392
2—Behera B K, Misra B N. (1983) Analysis of the effect of industrial effluent on pigments, proteins, nucleic acids, and the 2,6-dichlorophenol indophenol Hill reaction of rice seedlings. Environ Res. 31 (2): 381-389.
3—Maxwell K. Johnson G N (2000). Chlorophyll fluorescence—a practical guide. J. Exp. Bot. 51 (345): 659-668 Review
4—Zimmermann G. M., Kramer G. N., Schnabl H. (1996). Lyophilization of thylakoids for improved handling in a bioassay. Environ. Toxicol. Chem. 15(9): 1461-1463.
5—V. P. Torchilin, T. S. Levchenko, K. R. Whiteman, A. A. Yaroslavov, A. M. Tsatsakis, A. K. Rizos, E. V. Michailova, M. I. Shtilman (2001). Amphiphilic poly-N-vinylpyrrolidones: synthesis, properties and liposome surface modification. Biomaterials. Biomaterials 22: 3035-3044.
6—Shaker H (1982). Chemical Cryoprotection for structural studies. J. Microscopy 125 (2): 137-147.
7—Schreiber, U., Schliwa, W. and U. Bilger (1986). Continuous recording of photochemical and non-photochemical chlorophyll fluorescence quenching with a new type of modulation fluorimeter. Photosynthesis Research, 10, 51-62.
8—Conrad, R.; C. Büchel; C. Wilhelm; W. Arslane; C. Berkaloff and J.-C. Duval (1993). Changes in yield of in-vivo fluorescence of chlorophyll as a tool for selective herbicide monitoring. J. Appl. Phycol. 5: 505-516.
9—Porra R. J., Thompson W. A., Kriedemann P. E., (1989). Determination of accurate extinction coefficients and simultaneous equations for assessing chlorophylls a and b extracted with four different solvents: verification of the concentration of cholorophyll standards by atomic absorption spectroscopy. Biochimia Biophysica Acta 975: 384-394.
10—Butler, W. L., (1977). Chlorophyll fluorescence: a probe for electron transfer and energy transfer. In Encyclopaedia of Plant Physiology, Berlin: Springer-Verlag. ed. A. Trebst, M. Avron, 5, 149-167.
11—Butler, W. L., (1978). Energy distribution in the photochemical apparatus of photosynthesis. Annual Review of Plant Physiology, 29, 345-78.
12—Björkman, 0. and B. Demmig (1987). Photon yield of 02 evolution and chlorophyll fluorescence characteristics at 77K among vascular plants of diverse origins. Planta, 170, 489-504.
13—Laberge, Chartrand, Rouillon, Carpentier (1999). “In vitro phytotoxicity screening test using immobilized spinach thylakoids” Env. Tox. Chem. 18 (12): 2851-2858
14—Laberge, Rouillon, Carpentier (2000). “Comparative study of thylakoid membranes sensitivity for herbicide detection after physical or chemical immobilization” Enz. Microb. Technol. 26: 332-336
15—Koblizek, Maly, Masojidek, Komenda, Kucera, Giardi, Mattoo, Pilloton (2002). “A Biosensor for the Detection of Triazine and Polylurea Herbicides Designed Using Photosystem II Coupled to a Screen-Printed Electrode” Biotechnol. Bioeng. 78 (1): 110-116

1. A method of extracting active thylakoid membranes from a plant, the method comprising the steps of:
a) obtaining a plant tissue having Fv/Fm ratio of at least about 0.7;
b) disrupting the tissue in a medium having a viscosity between 1 and 1.3 and a pH above 5 and below 8 to obtain a mixture of cell debris and thylakoids in a liquid phase;
c) separating said debris from thylakoids;
d) suspending said thylakoids, and filtering to recover fractions;
e) pooling fractions with Fv/Fm greater than about 0.7; and
f) stabilizing said pooled fractions by adding polyvinylpyrrolidone (PVP) and removing water therefrom.

2. The method of claim 1, further comprising the steps of:
g) washing said tissue with sodium hypochlorite at about neutral pH;
h) conditioning said washed tissue under light conditions between 565 and 575 nm and at a temperature under about 10° C.;
i) disrupting said tissue as defined in claim 1;
j) separating said debris from thylakoids under centrifugation with an upper filter and recovering upper filter pellet essentially consisting of thylakoids;
k) suspending said pellet and filtering under Sephadex-G100, recovering and pooling fractions with Fᵥ/Fₘ greater than about 0.7; and
l) removing water from said pooled fractions by carrying out lyophilisation in a solution comprising polyvinylpyrrolidone (PVP) to recover said extract substantially free of electron donor;
wherein said substantially pure thylakoid extract comprises organized photosynthetic pigments selected from: chlorophyll A, chlorophyll B, lutein and carotene; wherein chlorophyll A is at a ratio of at least 0.6 of total pigment content;
whereby antioxidant activity of said substantially pure thylakoid extract is stable at room temperature for at least about 7 days.

3. The method of claim 1, wherein said antioxidant activity thylakoid extract is stable at 4° C. for at least about 2 months.

4. (canceled)

5. The method according to claim 1, wherein the suspension of step d) is obtained by mechanically dispersing said plant tissues in said medium.

6. The method according to claim 1, wherein said plant is spinach.

7. (canceled)

8. The method according to claim 1, wherein said viscosity is partly achieved by the presence of sorbitol in a concentration of about 0.2 to 0.4 M in said medium or of a sugar achieving a viscosity equivalent to 0.2 to 0.4 M sorbitol.

9. The method according to claim 1, wherein said medium has the following composition: Tris or acetate or ascorbate buffer (20 mM) having a pH of about 7.5 and 350 mM of sorbitol or sucrose.

10. The method of claim 5, wherein the step d) of separating comprises centrifuging the first extract in a tube equipped with a filter in a superior portion of the tube, the filter having a porosity onto which cell debris and membranes deposit while the thylakoids and the liquid phase pass through the filter, the thylakoids forming a pellet in an inferior portion of the tube.

11. The method of claim 10, wherein the removing water is carried out by adding about 0.5 to about 5% of polyvinylpyrrolidone (PVP) to said pooled fractions and carrying out lyophilization.

12. (canceled)

13. The method of claim 10, further comprising adding about 350 mM of sucrose or sorbitol to said pooled fractions prior to lyophilization.

14. (canceled)

15. (canceled)

16. The method of claim 1, wherein said substantially pure thylakoid extract is stable at 4° C. for about 1 year while maintaining at least about 70% of its original ORAC activity.

17. The method of claim 1, wherein said thylakoid extract has an original NO inhibition activity at day 0, whereby said substantially pure thylakoid extract is stable at 4° C. for at least about 1 year while maintaining at least 50% of its original NO inhibition activity when thylakoids are assessed at 0.25 mg/Ml.

18. (canceled)

19. A thylakoid extract made by the process of claim 1.

20. A composition comprising an active thylakoid extract, in admixture with PVP.

21. (canceled)

22. The composition of claim 20, wherein said PVP is present at about 0.5 to about 5% concentration.

23. The composition of claim 22, wherein said PVP is present at about 2% concentration.

24. (canceled)

25. The composition of claim 20, comprising from about 135 to about 165 μmol Trolox equivalent per g of thylakoid (μmol ET/g).

26. The composition of claim 20, comprising at least about 33% NO inhibition at a concentration at 0.25 mg/mL of thylakoid.

27. The composition of claim 20, comprising an SOD activity of at least about 0.4 mU/mg prot/min.
